(19) United States
(12) Patent Application Publication
(10) Pub. No.: US 2013/0295605 A1
(43) Pub. Date: Nov. 7, 2013

(54) METHOD FOR PRODUCING A POLYPEPTIDE IN YARROWIA LIPOLYTICA

(71) Applicant: Petrus Jakobus van Zyl, Pretoria (ZA)

(72) Inventor: Petrus Jakobus van Zyl, Pretoria (ZA)

(73) Assignee: CSIR, Pretoria (ZA)

(21) Appl. No.: 13/261,675

(22) PCT Filed: May 26, 2011
(86) PCT No.: PCT/IB2011/052303

§ 371 Date: Jun. 14, 2013

(30) Foreign Application Priority Data
May 28, 2010 (ZA) 2010/03839

(51) Int. Cl. C12P 21/00; C12N 15/81

(52) U.S. Cl. CPC C12P 21/00; C12N 15/815
USPC 435/69.1; 435/254.2

(57) ABSTRACT

This invention relates to a method for the production of proteins, preferably, heterologous proteins, under the regulation of the hp4d promoter, in Yarrowia lipolytica. In particular, this invention describes a method for manipulating the growth rate of Y. lipolytica by regulating the carbon and/or nitrogen supply. A growth rate of less than 0.045 h″l was found to be optimal for increasing Y. lipolytica biomass and increasing the amount of heterologous proteins of interest produced.

### BACKGROUND OF THE INVENTION

THIS invention relates to a method for producing a polypeptide in Yarrowia lipolytica. In particular, this invention relates to a method of optimizing the use of the hp4d promoter in expressing a polypeptide of interest in Y. lipolytica, by manipulating the growth conditions and hence the growth profile of the yeast.

Y. lipolytica is a non-conventional yeast which has been awarded Generally Regarded as Safe (GRAS) status by the American Food and Drug Administration (FDA) for citric acid production (Fickers et al., 2005). A large number of molecular tools are available for heterologous protein expression in Y. lipolytica as this yeast has a high secreting capacity. Multi-copy vectors contain the ura3d4 marker, which is required in multiple copies to complement, allowing for selection of transformants with multiple inserts (Madzak et al., 2004). The ura3d4 selection marker ensures selection of transformants with 10-13 copies of the integrated cassette (Juretzek et al., 2001).

The hp4d promoter is the most popular promoter used for expressing heterologous polypeptides in Y. lipolytica (Madzak et al., 2004). This promoter consists of four tandem repeat copies of the upstream activating sequence 1 of the XPR2 promoter, and expression is not significantly affected by environmental conditions. However, its regulation is unknown and it is reported to be quasi constitutive with production of proteins under its regulation only occurring during early stationary growth phase.

A need thus exists to gain a greater understanding of the hp4d promoter to determine whether this promoter will find application in manipulating, and ultimately increasing, protein expression in a host cell.

### SUMMARY OF THE INVENTION

According to the invention, there is provided a method of expressing a polypeptide in Yarowia lipolytica, the method comprising the steps of:
fermenting Y. lipolytica which has been transformed with a polynucleotide encoding the polypeptide under the control of a hp4d promoter; and
limiting the growth rate of the Y. lipolytica during fermentation to below 0.045 h⁻¹.

The growth rate may be limited to from about 0.023 h⁻¹ to about 0.040 h⁻¹, and more preferably from about 0.035 h⁻¹ to about 0.039 h⁻¹. Even more preferably, the growth rate may be limited to about 0.035 h⁻¹.

The growth rate may be limited by controlling the amount of a food source, such as a carbon and/or nitrogen source that is fed to the fermentation solution containing the Y. lipolytica. The carbon source may be glucose and the nitrogen source may be a yeast extract.

The polypeptide may be a protein such as an enzyme, for example, a lipase or mannanase.

The fermentation may be batch fermentation, fed batch fermentation, repeated fed batch fermentation or a continuous fermentation process.

Preferably, the method increases polypeptide production in comparison to a control Y. lipolytica whose growth rate was not limited.

In a further embodiment of the invention, there is provided Y. lipolytica, which has been transformed with a polynucleotide, encoding a polypeptide under the control of a hp4d promoter, for use in a method as described above.

In another embodiment of the invention, there is provided a kit comprising Y. lipolytica as described above for performing a method as described above.

### BRIEF DESCRIPTION OF THE FIGURES

FIG. 1: shows the effect of a glucose spike on the pO₂, residual glucose and biomass concentrations of steady state Y. lipolytica Po1f 413-5 fermentation.

FIG. 2: shows the effect of dilution rate on biomass, residual glucose and volumetric enzyme activity of Y. lipolytica Po1f 413-5 in steady state continuous fermentation.

FIG. 3: shows the effect of dilution rate on the magnitude of response in lipase production by Y. lipolytica Po1f 413-5 in steady state continuous fermentation. Growth rates are given as data labels.

FIG. 4: shows the effect of dilution rate on volumetric and specific rate of lipase production by Y. lipolytica Po1f 413-5 in steady state continuous fermentation.

FIG. 5: shows the growth of Y. lipolytica Po1f 413-5 in duplicate batch fermentation.

FIG. 6: shows lipase production by Y. lipolytica Po1f 413-5 in duplicate batch fermentation.

FIG. 7: shows the effect of exponential full medium feed on the growth of Y. lipolytica Po1f 413-5. Full medium was fed at exponential feed rates of 0.029 h⁻¹ (open symbols) and 0.041 h⁻¹ (closed symbols).

FIG. 8: shows the effect of exponential full medium feed on lipase production by Y. lipolytica Po1f 413-5. Full medium was fed at exponential feed rates of 0.029 h⁻¹ (open symbols) and 0.041 h⁻¹ (closed symbols).

FIG. 9: shows the effect of exponential full medium feed on the rate of lipase production by Y. lipolytica Po1f 413-5. Full medium was fed at exponential feed rates of 0.029 h⁻¹ (open symbols) and 0.041 h⁻¹ (closed symbols).

FIG. 10: shows growth of Y. lipolytica ManA:HmA (Roth et al., 2009) in duplicate batch fermentation.

FIG. 11: shows the effect of exponential full medium feed on the growth of Y. lipolytica ManA:HmA. Full medium was fed at exponential feed rates of 0.035 h⁻¹ (open symbols) and 0.045 h⁻¹ (closed symbols).

FIG. 12: shows the effect of exponential full medium feed on the mannanase production by Y lipolytica ManA:HmA. Full medium was fed at exponential feed rates of 0.035 h⁻¹ (open symbols) and 0.045 h⁻¹ (closed symbols)

FIG. 13: shows the effect of exponential full medium feed on the rate of mannanase production by Y lipolytica ManA:HmA. Full medium was fed at exponential feed rates of 0.035 h⁻¹ (open symbols) and 0.045 h⁻¹ (closed symbols).

### DETAILED DESCRIPTION OF THE INVENTION

The present invention will now be described more fully hereinafter with reference to the accompanying drawings, in which some, but not all embodiments of the invention are shown.

A method of expressing a polypeptide in Yarowia lipolytica is disclosed herein, wherein Y. lipolytica, which has been transformed with a polynucleotide encoding the polypeptide under the control of a hp4d promoter, is fermented and the growth rate of the Y. lipolytica is limited during fermentation to below 0.045 h⁻¹.

The growth rate can be limited to from about 0.023 h⁻¹ to about 0.040 h⁻¹, and more preferably from about 0.035 h⁻¹ to about 0.039 h⁻¹. For example, the growth rate can be limited to about 0.023, 0.024, 0.027, 0.029, 0.035 or 0.039 h⁻¹.

The growth rate can be limited by controlling the amount of a carbon, nitrogen and/or other source, such as glucose or yeast extract, that is fed to the fermentation solution.

The fermentation may be batch fermentation, batch fed fermentation or a continuous fermentation process.

The polypeptide can be a heterologous or homologous polypeptide, protein or enzyme. In the examples described below, lipase and mannanase were used as exemplary enzymes expressed by Y. lipolytica.

Production of enzymes by Y. lipolytica under regulation of the quasi-constitutive hp4d promoter occurs from the beginning of the stationary growth phase. Continuous fermentation under glucose limited conditions was used to determine the effect of growth rate on lipase produced under regulation of the hp4d promoter.

The Lip2 gene, encoding an extracellular lipase in Y. lipolytica, and endo-1,4-β-D-mannanase (β-mannanase) from Aspergillus aculeatu were over-expressed in Y lipolytica Polf (MatA, Leu2-207, ura3-302, xpr2-322, axp-2) with a multi-copy expression cassette of LIP2 under the quasi-constitutive hp4d promoter.

The highest volumetric lipase production of 13 014 nkat·ml⁻¹ was at a growth rate of 0.024 h⁻¹, the slowest growth rate evaluated. However, the maximum rate of lipase production was obtained at growth rates above 0.035 h⁻¹. The critical growth rate for lipase production was found to be between 0.035 h⁻¹ and 0.039 h⁻¹. The specific rate of lipase production of 28 nkat·mg⁻¹·h⁻¹ in continuous fermentation was 4 fold higher than the specific rate of lipase production of 7 nkat·mg⁻¹·h⁻¹ in batch fermentation, indicating that continuous fermentation may be a feasible option for enzyme production by Y. lipolytica. Utilizing the data obtained from the continuous fermentation, a fed batch strategy for protein production by Y. lipolytica under regulation of the hp4d promoter was developed and evaluated for the production of lipase and mannanase.

A maximum lipase titre of 22 508 (±4 219) nkat·ml⁻¹ was obtained when the growth rate during the fed batch phase of the fermentation was 0.027 h⁻¹ compared to 8 374 (±671) nkat·ml⁻¹ obtained at the higher growth rate of 0.040 h⁻¹ and 5 910 (±524) nkat·ml⁻¹ in batch fermentation. By limiting the growth rate of Y lipolytica we were able to achieve simultaneous biomass and enzyme production, thereby increasing the productivity of the fermentation. The volumetric lipase productivity was 357 nkat·ml⁻¹·h⁻¹ during the slower growth rate compared to 133 nKat·ml⁻¹·h⁻¹ during an exponential growth rate of 0.040 h⁻¹.

A maximum mannanase titre of 40 835 (±2 536) nkat·ml⁻¹ was obtained when the medium was fed exponentially at 0.035 h⁻¹ compared to 31 479 (±1 819) nkat·ml⁻¹ when the medium was fed at an exponential feed rate of 0.045 h⁻¹ and 14 253 (±2 807) nkat·ml⁻¹ in batch fermentation. The exponential feed strategy allowed for combined biomass and enzyme production, thereby increasing the productivity of the fermentation. The volumetric enzyme productivity was 913 nkat·ml⁻¹·h⁻¹ during the slower feed rate compared to 850 nkat·ml⁻¹·h⁻¹ and 346 nkat·ml⁻¹·h⁻¹ during an exponential feed rate of 0.045 h⁻¹ and batch production respectively. This feeding strategy was evaluated using a carbon feed as an example.

The invention as described should not to be limited to the specific embodiments disclosed and modifications and other embodiments are intended to be included within the scope of the invention.

Although specific terms are employed herein, they are used in a generic and descriptive sense only and not for purposes of limitation. The term “protein” for example, should be read to include “peptide” and “polypeptide” and vice versa. Furthermore, by definition protein includes “enzymes”.

### EXAMPLES

### Materials and Methods

### Organism Maintenance and Inoculum Strains

Y. lipolytica Po1f 413-5 (MatA, Leu-2-207, ura3-302, xpr2-322, axp-2) and Y lipolytica ManA:HmA (Roth et al., 2009) were cryo-preserved and stored at −80° C. The inoculum for fermentation was prepared by sterilising 100 ml medium consisting of 15 g·l⁻¹ yeast extract, 8.9 g·l⁻¹ malt extract and 6.67 g·l⁻¹ glucose in 1 L Erlenmeyer flasks at 121° C. for 15 min. The pH was be adjusted to 5.5 with either 25% m·v⁻¹ NH₄OH or 25% m·v⁻¹ H₂SO₄ before sterilisation. The content of single cryovials was used to inoculate the flasks. The flasks were incubated at 30° C. on an orbital shaker at 180 rpm for 18 h.

Continuous Fermentation of Y. lipolytica

A 2 L continuous fermenter (BioFlo 3000, New Brunswick, USA,) containing 1.5 L modified CSIRman medium consisting of 20 g·l⁻¹ yeast extract and 20 g·l⁻¹ glucose was inoculated with 100 ml inoculum. The pH was controlled at pH 6.8 with NH₄OH (25% m·v⁻¹) or H₂SO₄ (25% m·v⁻¹). The temperature was controlled at 28° C., aeration at 3 slmp and agitation at 800 rpm. Cal Biomass and optical density (OD) were measured by taking 5 ml samples after every retention time to determine steady state. Once steady state was reached as indicated by constant OD and biomass for three retention times, a sample was taken and biomass, OD, enzyme activity and residual glucose concentration was measured.

### Batch and Fed-Batch Lipase Fermentations

Duplicate batch fermentations were run in Labfors (Infors AG-Bottmingen/Switzerland) bioreactors with a working volume of 2 L containing 1.5 L medium consisting of 20 g·l^{1 -}yeast extract and 40 g·l⁻¹ glucose. Fed-batch fermenters were run in the same fermenters with an initial charge volume of 1.3 L consisting of 10 g·l⁻¹ yeast extract and 24 g·l⁻¹ glucose. The fermenters were inoculated with 100 ml inoculum. The pH was controlled at pH 6.8 with 25% m·v−1 NH₄OH or 20% m·v⁻¹ H₂SO₄. The temperature was controlled at 28° C. and the aeration set to 1 v·v⁻¹·m⁻¹. The starting agitation was 500 rpm and ramped up manually to control the pO₂ above 30% saturation. The feed consisted of 83.6 g·l⁻¹ glucose and 40 g·l⁻¹ yeast extract. The feed was started at depletion of the initial charge glucose as determined by Accutrend (Boehringer Mannheim). The starting feed rate was 1.1 g·h⁻¹ and increased every ten seconds at an exponential rate of 0.029 h⁻¹ and 0.041 h⁻¹, respectively.

### Analysis

Growth rate, biomass, enzyme production and glucose utilization were determined by taking 10 g samples at 3 hourly intervals. Growth was measured by determining the OD at 660 nm and the residual glucose was measured using Accutrend (Boehringer Mannheim). Triplicate samples of 2 ml aliquots were centrifuged and the supernatants stored at −20° C. for analyzes of extracellular lipase activity. The pellets were used for dry cell weight determination by drying to constant weight at 110° C.

The substrate for lipase assay was prepared by drop wise addition of 1 ml 8 mM p-Nitrophenylpalmitate (pNPP) prepared in isopropanol to 9 ml of 100 mM phosphate or Tris-HCl buffer, pH 8.0. The reaction was initiated by adding 25-50 μl of the enzyme sample and the release of pNP was monitored at 410 nm at 37° C. The activity of the enzyme was calculated as:

U·ml⁻¹=(V/v×ε×d)×A/min
where
   V=final volume,
   v=sample volume,
   ε=Extinction coefficient of p-NP at 410 nm,
   pH 8.0 (15 Mol⁻¹×cm⁻¹=ml×μmol⁻¹×cm⁻¹),
   d=light path of cuvette and
   A·min⁻¹=change of absorbance per minute.

The activity of the mannanase enzyme produced was determined by using 0.25% galactomannan (Sigma) in 0.05 M citrate phosphate buffer, as described by Bailey et al., (1992). The amount of reducing sugars released during the degradation of mannan was determined by the dinitrosalicylic acid method using mannose as standard (Miller et al., 1960). One unit of enzyme was defined as the activity producing 1 mmol reducing sugar per minute in mannose equivalents under the optimal assay conditions. Volumetric enzyme activity was reported as unit of enzyme per ml fermentation broth while the specific enzyme activity was reported as enzyme unit per mg dry cell weight in the fermentation broth.

### Results and Discussion

### Continuous Fermentation

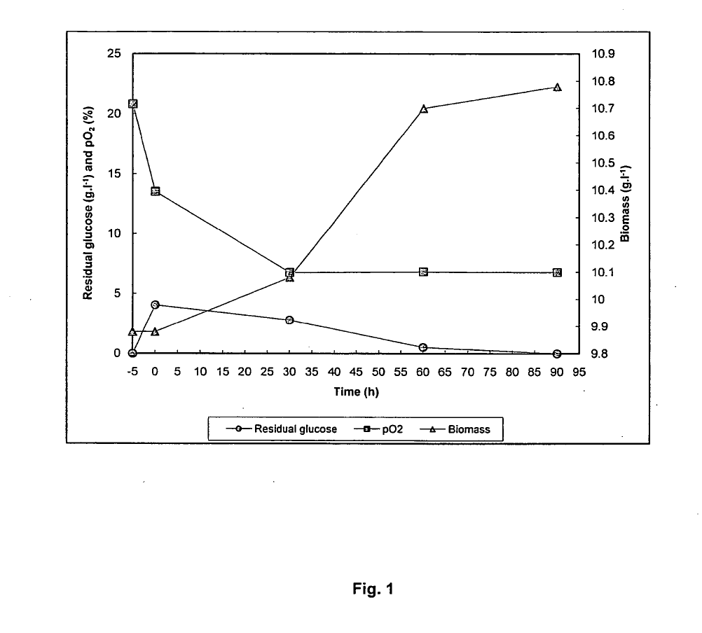

The effect of growth rates between 0.024 h⁻¹ and 0.058 h⁻¹ on the production of biomass and lipase enzyme were evaluated in continuous fermentation. Glucose was determined to be the growth limiting nutrient by spiking the fermenter with a concentrated glucose solution (50% m/m) to obtain a final glucose concentration in the fermenter of 5 g·l⁻¹. The pO2 decreased immediately in response to the glucose spike and the biomass increased from 9.9 g·l⁻¹ to 10.8 g·l¹ over 1.5 hours (FIG. 1).

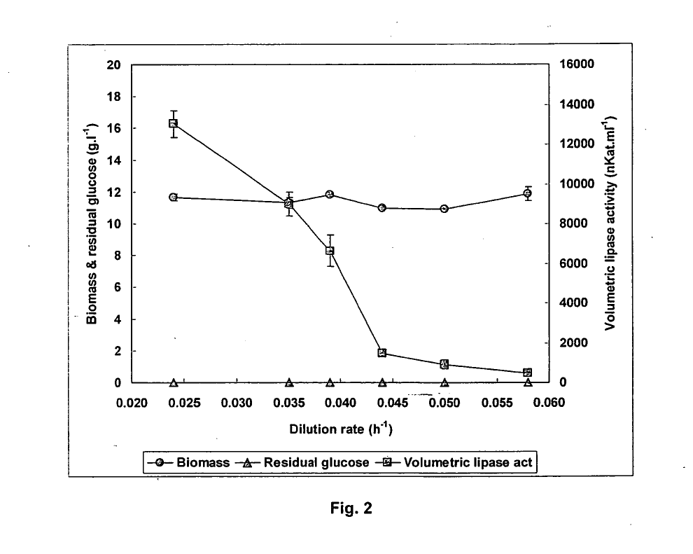

A constant biomass of 11.4 (±0.4) g·l⁻¹ was maintained at the dilution rates evaluated and the residual glucose remained below detection levels (FIG. 2). The highest volumetric enzyme activity of 13 014 nkat·ml⁻¹ was obtained at the slowest dilution rate of 0.024 h⁻¹. The highest dilution rate evaluated was 0.058 h⁻¹ with a volumetric enzyme of 493 nkat·ml⁻¹.

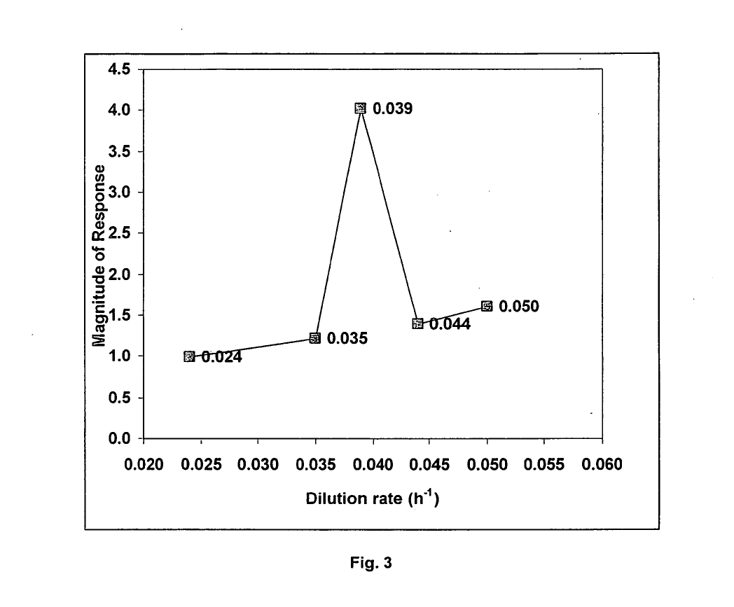

The magnitude of the response in lipase production as a result of increasing growth rate was calculated by dividing the fold decrease in enzyme activity by the fold increase in dilution rate. The effect of increased growth rate on lipase production was the highest when the growth rate was increased from 0.039 h⁻¹ to 0.044 h⁻¹, resulting in a magnitude of response of 4 indicating a critical growth rate for lipase production under regulation of the hp4d promoter by Y. lipolytica Po1f 413-5 (FIG. 3).

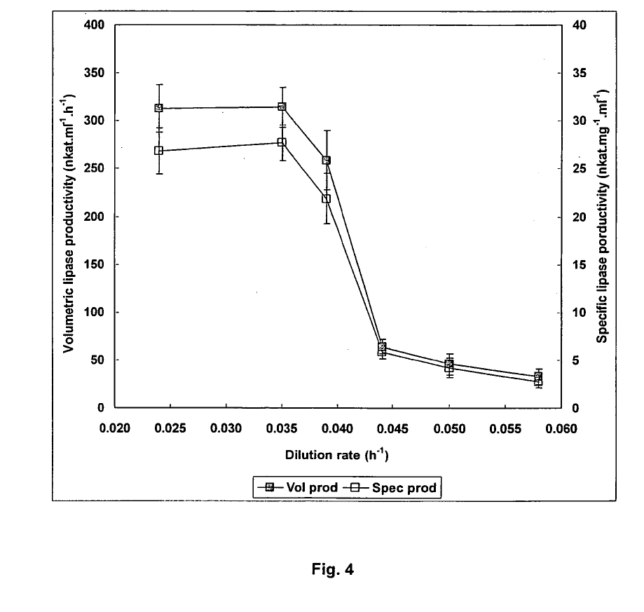

Both the volumetric and specific rate of lipase production were at their highest at dilution rates of 0.024 h⁻¹ and 0.035 h⁻¹ at of 314 nkat·ml⁻¹·h⁻¹ and 28 nkat·mg·h⁻¹, respectively (FIG. 4). Increasing the dilution rate above 0.035 h⁻¹ resulted in a decrease in the rate of lipase production, with a sharp drop in the rate of production when the dilution rate was increased above 0.039 h⁻¹. At a dilution rate of 0.044 h⁻¹, the volumetric rate of lipase production was 1 463 nkat·ml⁻¹·h⁻¹ and the specific rate of production was 6 nkat·mg⁻¹·h⁻¹. This drop in lipase productivity at growth rates above 0.039 h⁻¹ supports the results indicating that the hp4d promoter is regulated by growth rate and that it is fully expressed at growth rates slower than 0.035 h⁻¹, with 0.039 h⁻¹ being the critical growth rate for regulation of the hp4d promoter.

The maximum specific rate of lipase production in continuous fermentation was 28 nkat·mg⁻¹·h⁻¹ compared to 7 nkat·mg⁻¹·h⁻¹ in batch fermentation, making continuous fermentation a feasible option for enzyme production.

To utilise the effect of growth rate on lipase production, a fed batch strategy from lipase production by Y. lipolytica Po1f 413-5 was determined. The yield of biomass on glucose was determined to be 0.59 g·g⁻¹. From the data, a glucose feed rate for enzyme production in fed-batch fermentations was calculated using the following formula:

((dcwₜ₀+(dcwₜ₀×((2/td)))−dcwₜ₀)×Yx/s)/dcwₜ₀
where
   dcwₜ₀=starting dcw
   td=doubling time
   Yx/s=yield

At a growth rate of 0.039 h⁻¹, the calculated glucose feed rate was 0.07 g·g⁻¹·h⁻¹.

This feed rate was tested in fed-batch fermentations.

### Batch Fermentation

### Lipase Fermentation

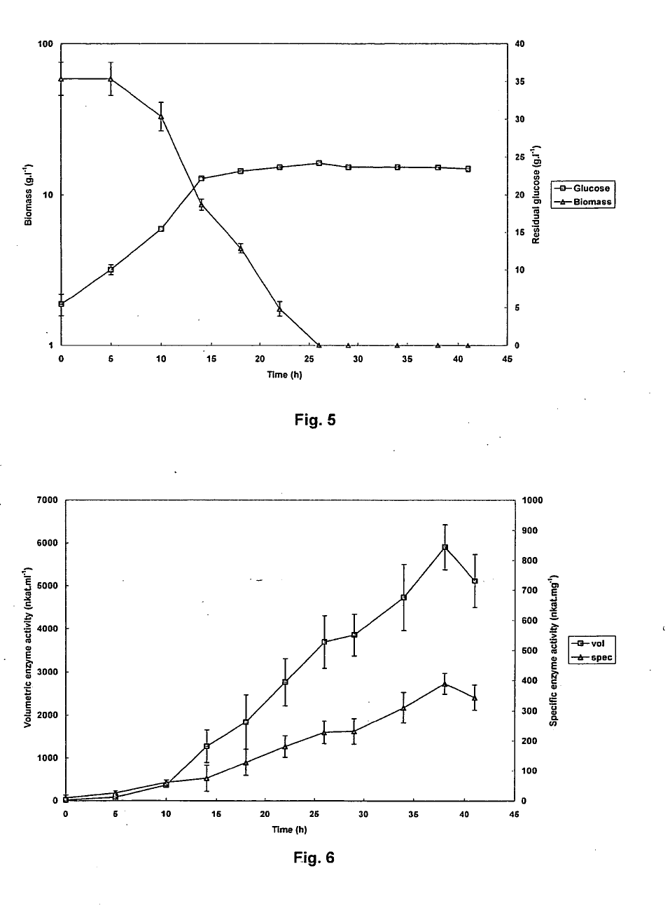

Batch fermentations were run to determine the production of the lipase enzyme under regulation of the hp4d promoter by Y. lipolytica Po1f 413-5 at maximum growth rates. Y. lipolytica Po1f 413-5 grew at a maximum growth rate of 0.14 and reached a biomass concentration of 16 (±0.36) g·l⁻¹ after 26 h (FIG. 5). The yield of biomass on glucose consumed was 0.67 g·g⁻¹.

The volumetric lipase produced by the end of the exponential growth phase was 1 274 (±377) nkat·ml⁻¹ and increased 4.6 fold to a maximum lipase activity of 5 910 (±524) nkat·ml⁻¹ after 38 hours (FIG. 6). The specific lipase production followed a similar trend and at the end of exponential growth the specific lipase was 75 (±44) nkat·mg⁻¹ but increased 5 fold over the next 24 hours to 390 (±35) nkat·mg⁻¹.

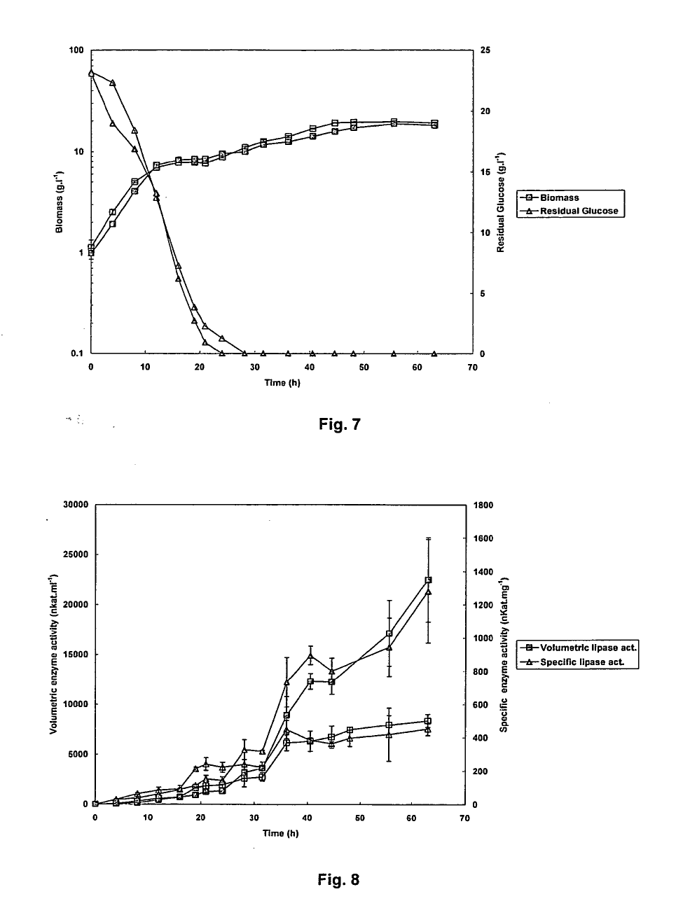

A fed-batch strategy consisting of a full medium feed was used to limit the growth rate. The feed was started after glucose depletion. The maximum growth rate during the batch phase was 0.13 (±0.01) h⁻¹ (FIG. 7).

The exponential growth rates after feed start were 0.027 h⁻¹ and 0.40 h⁻¹ for medium fed at exponential feed rates of 0.029 h⁻¹ and 0.041 h⁻¹, respectively. During the batch phase of the fermentation, 724 (±13) nKat·ml⁻¹ lipase was produced. The majority of the lipase was produced during the exponential feed period, with 7 545 (±246) nKat·ml⁻¹ lipase produced during growth at 0.40 h⁻¹ compared to 17 152 (±410) nKat·ml⁻¹ lipase during growth at 0.027 h⁻¹ (FIG. 8).

The maximum volumetric lipase activity achieved during growth at the slower growth rate was 2.7 fold higher at 22 508 (±4219) nKat·ml⁻¹ than the maximum lipase activity of and 8 374 (±671) nKat·ml⁻¹ obtained at the higher growth rate. The response of the specific lipase activity to the growth rate was similar to that of the volumetric lipase activity, with a 2.8 fold higher activity obtained at the lower growth rate. The maximum specific lipase activity was 1 281 (±311) nkat·mg⁻¹ at a growth rate of 0.027 h⁻¹ compared to 452 (±352) nkat·mg⁻¹ at a growth rate of 0.040 h⁻¹.

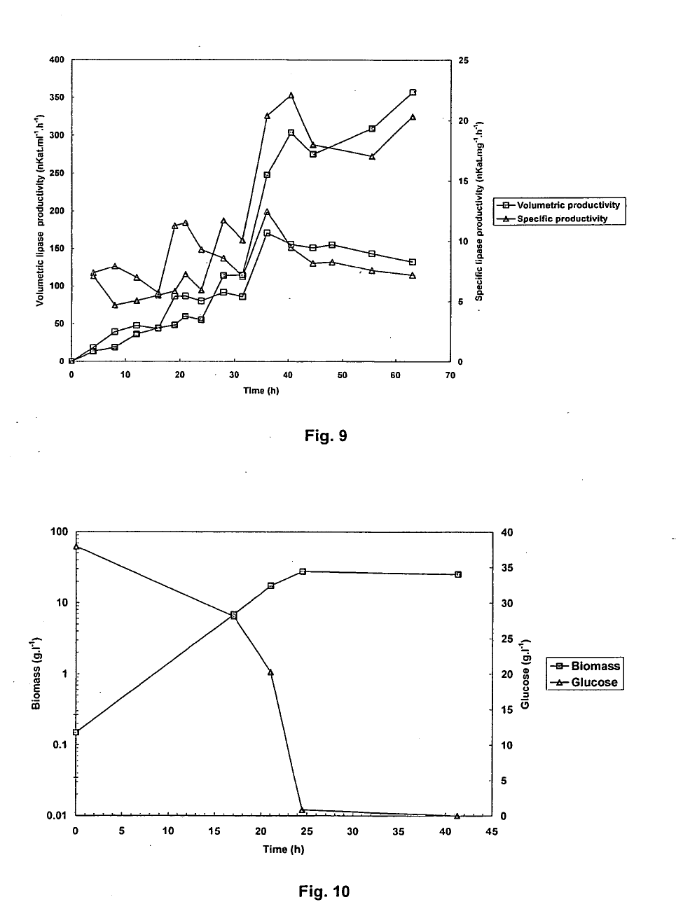

The productivity was not influenced by the slower growth rate, and maximum volumetric and specific productivities of 357 nkat·ml⁻¹·h⁻¹ and 20.3 nkat·mg⁻¹·h⁻¹ were obtained at a growth rate of 0.027 h⁻¹. At a growth rate of 0.040 h⁻¹, the volumetric productivity was 133 nKat·ml⁻¹·h⁻¹ and specific productivity was 7.2 nkat·mg⁻·h⁻¹ (FIG. 9).

### Mannanase Fermentation

Batch fermentations were run to determine the production of the mannanase enzyme under regulation of the hp4d promoter by Y lipolytica at maximum growth rates. Y lipolytica grew at a maximum growth rate of 0.23 h⁻¹ and reached a biomass concentration of 27 (±0.74) g·l⁻¹ after 25 h at a yield of 0.68 g·g⁻¹ glucose (FIG. 10). Mannanase activity can only be determined after glucose depletion since residual glucose interferes with the enzyme assay. The mannanase was therefore determined at the point of glucose depletion and again 16 hours later. However, the activity at the point of glucose depletion is the most important, as it provides an indication of the amount of enzyme that is produced during unlimited growth rate. The volumetric and specific mannanase activity was 10 427 (±967) nkat·ml⁻¹ and 386 (±13) nkat·mg⁻¹, respectively, at glucose depletion but increased to 14 253 (±2 807) nkat·ml⁻¹ and 527.9 (±88) nkat·mg⁻¹ during the next sixteen hours.

Using a fed-batch strategy to limit the maximum growth rate, a full medium feed, with glucose as the limiting nutrient as determined in continuous fermentation, was started at glucose depletion (after 16 h) at a rate of 0.1 g(glucose)·l⁻¹·h⁻¹ and increased at exponential rates of 0.035 and 0.045 h⁻¹, respectively (FIG. 11). The growth rate during the batch phase was 0.24 h⁻¹ reaching a biomass of 15 (±0.3) g·l⁻¹ at a yield of 0.63 g·g⁻¹. The exponential feed rates employed limited the growth rates during the fed-batch phase of the fermentations to 0.033 h⁻¹ and 0.044 h⁻¹, respectively, and a final biomass of 28 (±0.6) g·l⁻¹ was obtained at the end of the fed batch phase.

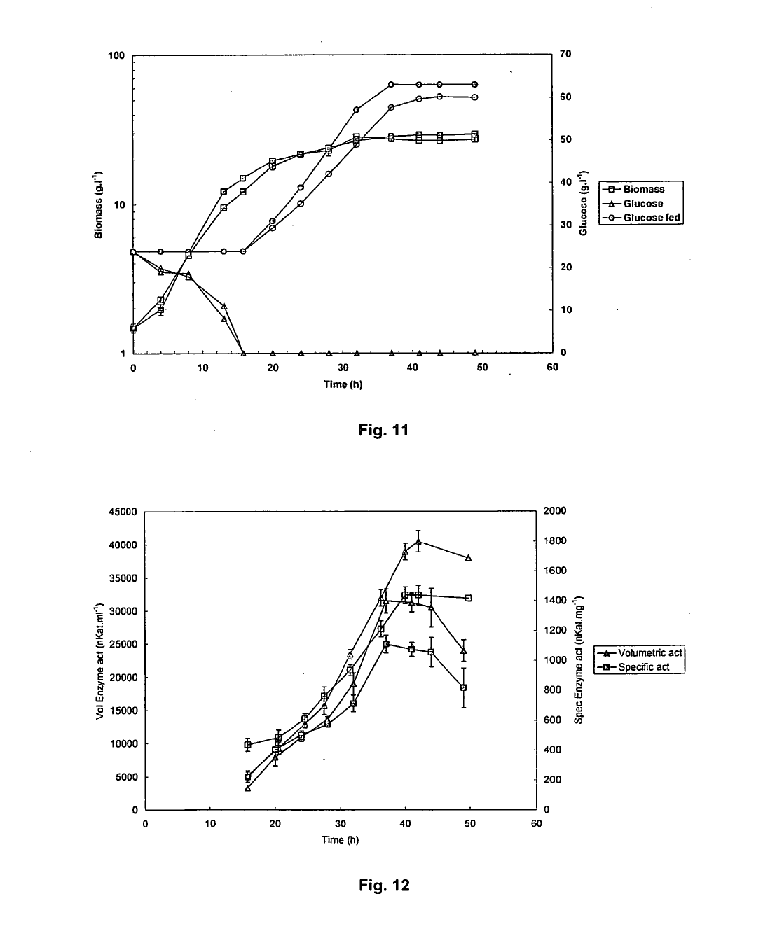

The average volumetric and specific mannanase activities, for duplicate fermentations with triplicate assays, at the end of the batch phase were 5 211 (±602) nkat·ml⁻¹ and 436 (±47) nkat·mg⁻¹, respectively (FIG. 12). The maximum volumetric enzyme activity produced when Y. lipolytica was fed medium at an exponential rate of 0.035 h⁻¹ was 40 480 (±1 268) nkat·ml⁻¹ compared to 31 479 (±1 819) nkat·ml⁻¹ when the medium was fed at an exponential rate of 0.045 h⁻¹.

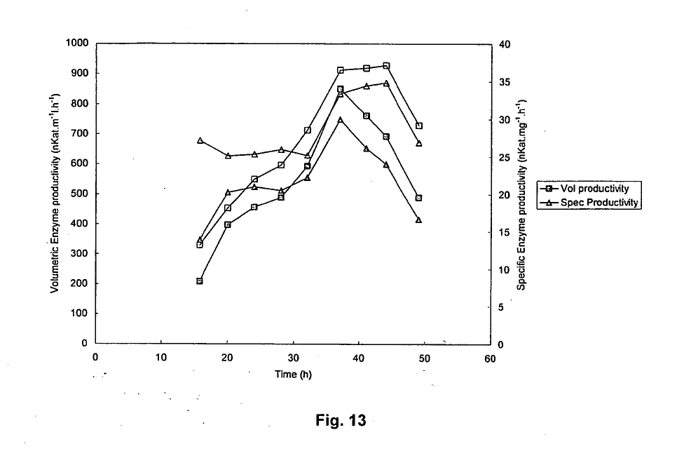

The specific enzyme activity increased 1.4 fold, from 1 109 (±60) nkat·mg⁻¹ when the medium was fed at an exponential feed rate of 0.045 h⁻¹ to 1 533 (±83) nkat·mg⁻¹ when the fermenter was fed at an exponential rate of 0.035 h⁻¹. The slower feed rate did not result in slower productivity of the mannanase and the enzyme was produced at 913 nkat·ml⁻¹·h⁻¹ at exponential feed rates of 0.035 h⁻¹ and 0.045 h⁻¹ (FIG. 13). This was 2.6 fold higher than the productivity of 346 nKat·ml⁻¹·h⁻¹ achieved in batch fermentation.

### CONCLUSION

This is the first reported data on the heterologous production of enzymes by Y. lipolytica in continuous fermentation. The critical growth rate for enzyme production was found to be between 0.035 h⁻¹ and 0.039 h⁻¹, and from this data a glucose feed rate for fed-batch fermentations could be calculated. This feed strategy was evaluated in fed batch fermentation for production of enzymes under regulation of the hp4d promoter in Y. lipolytica.

To evaluate the effect of growth rate on lipase production, two fed batch fermentations were run with a full medium exponential feed based on the glucose concentration of the feed at rates of 0.029 h⁻¹ and 0.041 h⁻¹, resulting in growth rates of 0.027 h⁻¹ and 0.040 h⁻¹, respectively. The volumetric and specific enzyme activities were 3.8 and 3.3 fold higher when the growth rate was limited 0.027 h⁻¹ compared to batch fermentation. The volumetric lipase activity at a growth rate of 0.027 h⁻¹ was 1.7 fold higher than that obtained at a growth rate of 0.040 h⁻¹. This compares favourably with the 1.9 fold increase in volumetric lipase activity obtained in continuous fermentation when the growth rate was decreased from 0.039 h⁻¹ to 0.024 h⁻¹.

The volumetric and specific mannanase activities were 2.7 and 2.9 fold higher when the growth rate was limited by the exponential feed of 0.035 h⁻¹ compared to batch fermentation. The ability to maximize the specific enzyme activity utilizing an exponential feed rate below 0.045 h⁻¹ can be exploited by decreasing the feed rate from 0.056 h⁻¹ to 0.035 h⁻¹ once a high biomass concentration has been reached. This will allow for high specific and volumetric enzyme production under regulation of the hp4d promoter in Y lipolytica.

The data presented in this report shows that the production of enzymes under regulation of the hp4d promoter can be switched on during biomass production by limiting the growth rate using an exponential feed strategy.

### REFERENCES

Bailey M J, Biely P, Poutanen K (1992) Interlaboratory testing of methods for assay of xylanase activity. J Biotechnol 23: 257-270
Fickers P, Benetti P-H, Wache Y, Marty A, Mauersberger S, Smit M S, Nicaud J-M (2005) Hydrophobic substrate utilization by the yeast Yarrowia lipolytica, and its potential applications. FEMS Yeast Res 5: 527-543.
Juretzek T, Le Dall M, Mauersberger S, Gaillardin C, Barth G, Nicaud J-M (2001) Vectors for the expression and amplification in the yeast Yarrowia lipolytica. Yeast 18, 97-113.
Madzak C, Gaillardin C, Beckerich J-M (2004) Heterologous protein expression and secretion in the non-conventional yeast Yarrowia lipolytica. J Biotechnol 109: 63-81.
Miller G L, Blum R, Glennon W E, Burton A L (1960) Measurement of carboxymethylcellulase activity. Anal Biochem 2: 127-132
Roth R, Moodley V and P van Zyl. (2009) Heterologous Expression and Optimized Production of an Aspergillus aculeatus Endo-1,4-β-mannanase in Yarrowia lipolytica. Mol. Biotechnol. 43: (2) 112-120.

1. A method of expressing a polypeptide in Yarrowia lipolytica, the method comprising the steps of:
fermenting Y. lipolytica which has been transformed with a polynucleotide encoding the polypeptide, under the control of a hp4d promoter; and
limiting the growth rate of the Y. lipolytica during fermentation to below 0.045 h⁻¹.

2. The method of claim 1, wherein the growth rate is limited in the range of about 0.023 h⁻¹ to about 0.040 h⁻¹.

3. The method of claim 2, wherein the growth rate is limited in the range of about 0.035 h⁻¹ to about 0.039 h⁻¹.

4. The method of claim 3, wherein the growth rate is limited to about 0.035 h⁻¹.

5. The method of claim 1, wherein the growth rate is limited by controlling the amount of a food source that is fed to a fermentation solution containing the Y lipolytica.

6. The method of claim 5, wherein the food source is a carbon and/or nitrogen source.

7. The method of claim 1, wherein fermentation is carried out via any one of batch fermentation, fed batch fermentation, repeated fed batch fermentation or continuous fermentation.

8. The method of claim 7, wherein the fermentation is fed batch fermentation.

9. The method of claim 7, wherein the fermentation is repeated fed batch fermentation.

10. The method of claim 7, wherein the fermentation is continuous fermentation.

11. The method of claim 1, in which the level of expressed polypeptide production is increased in comparison to a control Y. lipolytica whose growth rate was not limited.

12. Y. lipolytica, which has been transformed with a polynucleotide encoding a polypeptide under the control of a hp4d promoter, for use in a method according to claim 1.

13. A kit comprising Y. lipolytica according to claim 12 for performing a method according to claim 1.
